(19) United States
(12) Patent Application Publication
(10) Pub. No.: US 2008/0306575 A1
(43) Pub. Date: Dec. 11, 2008

(54) Body Tanning Device

(76) Inventor: Roberto Schiavon, Cadoneghe (IT)

Correspondence Address: Modiano & Associati, Via Meravigli, 16, Milano, 20123, IT

(21) Appl. No.: 11/885,995

(22) PCT Filed: Mar. 10, 2006
(86) PCT No.: PCT/EP2006/060624

§ 371 Date: Sep. 10, 2007

(30) Foreign Application Priority Data
Mar. 31, 2005 (IT) PD2005U000020

(51) Int. Cl. A61N 5/06

(52) U.S. Cl. 607/91

(57) ABSTRACT

A body tanning device, comprising: a chassis for support on the floor and/or on a wall; a supporting frame for a tanning ray source, which has a large opening for the exit of the tanning rays, the frame being pivoted to the chassis in order to assume at least two stable positions: a first position for use, which is substantially horizontal, and a second substantially vertical position for nonuse; elements for moving and supporting the frame from the first stable position to the second stable position and vice versa; a retractable covering, substantially of the fold-away type, for the tanning ray source, the covering being associated with the frame.

### TECHNICAL FIELD

The present invention relates to a body tanning device.

### BACKGROUND ART

As it is known, body tanning devices are divided substantially into two types: the full-body type, which is intended to tan the entire body, or the partial type, which tans only one part of the body, typically the face.

With reference to full-body tanning devices, two different categories of apparatus are known and differ in terms of the position assumed by the body of the user during application.

These apparatuses are known as tanning booths when the user stands up.

These apparatuses are instead known as tanning beds when the user lies down.

Tanning booths are constituted by two panels which contain sources of tanning rays, which are arranged vertically and in front of each other.

Both flat and curved panels are known; their use is determined by the construction requirements of the individual tanning device.

A tanning source is constituted by one or more tanning lamps arranged side-by-side, such as for example low-pressure fluorescent lamps or a high-pressure mercury-vapor lamps.

In tanning beds, the panels which surround the user are arranged horizontally; the upper one is generally known as canopy and the bottom one continues to be known as bed, since it is used as a support for the person.

In some tanning beds, the tanning sources can be inserted in both panels; in this manner, the user can tan both in the lower part and in the upper part of the body.

In other kinds of tanning bed, the tanning source is inserted only in the upper panel (tanning canopy) and the lower panel acts only as a support for the person; in this case, the tanning applications must be performed first on one side of the body and then on the other.

Other types of tanning device are provided only with the upper part (tanning canopy), which is pivoted to a supporting frame and are known as tanning canopies; in this case, a supporting surface for the user must be associated with the tanning device.

Wheels are generally associated with the frame of the device in order to move the device from an area for nonuse to an area for use, such as for example the home bed.

Generally, the panels of these devices are pivoted to the chassis and are provided with supporting means, such as gas-assisted struts, which are arranged downward and laterally with respect to the panels; such gas-assisted struts are designed to support the panels in a horizontal position and to allow a more or less facilitated movement thereof from a vertical position to a horizontal position and vice versa.

Devices are also known which are constituted by a single panel, which is typically arranged vertically (and in this case the user must stand in order to tan) and is associated with wall supporting means.

An example of a tanning panel of this type is disclosed in Italian Patent Application No. VR2000A000025.

This patent application describes a panel with a tanning ray source which is fixed to a supporting structure.

A further covering panel is associated with the tanning panel and is hinged to the panel like the door of a piece of furniture.

The purpose of this additional panel is to hide from sight the tanning source and therefore make it pleasant in a home environment.

Such covering panel is provided for example by means of a mirror.

The same patent application also illustrates a configuration in which the tanning panel is pivoted to the wall and can be moved from a position in which it is adjacent to the wall to a position in which it is perpendicular thereto, so as to allow a user arranged horizontally to tan.

In this case, brackets for supporting the tanning panel are associated below the panel, in order to prevent the panel from falling onto the user.

As described, some of the solutions mentioned above allow to use, as a support for the user, normal bed-like furniture usually available in a domestic environment, such as a couch, a bed, a deckchair or others.

These kinds of device, therefore, can be converted in order to allow to close them and move them to a position which is different from the position in which they are used.

However, these devices are extremely bulky and their conversion and movement operations are not trivial and are also laborious.

The type of tanning source that is used in these devices is, for tanning canopies, typically constituted by low-pressure fluorescent lamps, substantially composed of fluorescent lamps with a length of 180 to 200 centimeters.

At present, this type of tanning technology is the one which ensures the best tanning for an equal electrical power consumption, and is therefore preferred.

The light-emitting surface of devices which use low-pressure fluorescent lamps is very large and therefore the tanning panel is consequently quite heavy.

The weights of the panel are in practice not negligible and make it difficult and dangerous to move the panel from a vertical position to a horizontal position and vice versa and to move it from one room to another.

The presence of gas-assisted struts or of the chassis in the region below the tanning panel is problematic, since they interfere with the edge of the bed (bed, couch or other bed-like furniture) with which the tanning device is associated.

### DISCLOSURE OF THE INVENTION

The aim of the present invention is to provide a body tanning device which solves the drawbacks noted above in conventional devices.

Within this aim, an object of the present invention is to provide a body tanning device which can be converted safely from a position for use to a position for nonuse and vice versa, and can be used conveniently in any environment (home, workplace or other) without having to perform particular and awkward conversions of such environment.

Another object of the present invention is to provide a body tanning device which has smaller dimensions than normally commercially available devices.

Another object of the present invention is to provide a body tanning device which can have additional functions besides simple tanning.

A further object of the present invention is to provide a body tanning device which can be used in full safety both as regards tanning and as regards setting it up.

This aim and these and other objects, which will become better apparent hereinafter, are achieved by a body tanning device, which comprises:
a chassis for support on the floor and/or on a wall,
a supporting frame for a tanning ray source, which has a large opening for the exit of said tanning rays, said frame being pivoted to said floor and/or wall supporting chassis in order to assume at least two stable positions: a first position for use, which is substantially horizontal, and a second substantially vertical position for nonuse,
means for moving and supporting said frame from said first position to said second position and vice versa,

characterized in that it comprises a retractable covering, substantially of the fold-away type, for said tanning ray source, said covering being associated with said frame.

### BRIEF DESCRIPTION OF THE DRAWINGS

Further characteristics and advantages of the invention will become better apparent from the following detailed description of a preferred but not exclusive embodiment thereof, illustrated by way of non-limiting example in the accompanying drawings, wherein:

FIG. 1 is a schematic front view of a device according to the invention, fixed to a wall above a couch;

FIG. 2 is a schematic side view of the device according to the invention, arranged in the position for nonuse;

FIG. 3 is a schematic side view of the device according to the invention, arranged in the position for nonuse;

FIG. 4 is an enlarged-scale view of a detail of FIG. 3;

FIG. 5 is an exploded view of the supporting chassis of the device according to the invention;

FIG. 6 is a view of a detail of the device according to the invention, related to safety means for preventing the fall of the frame that supports the tanning ray source;

FIG. 7 is a view of the detail of FIG. 6, with the fall prevention safety means active.

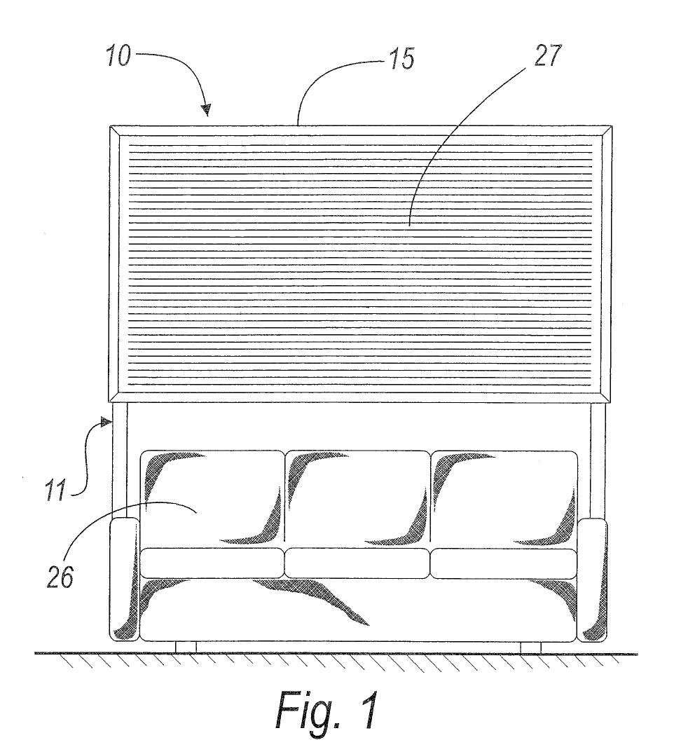

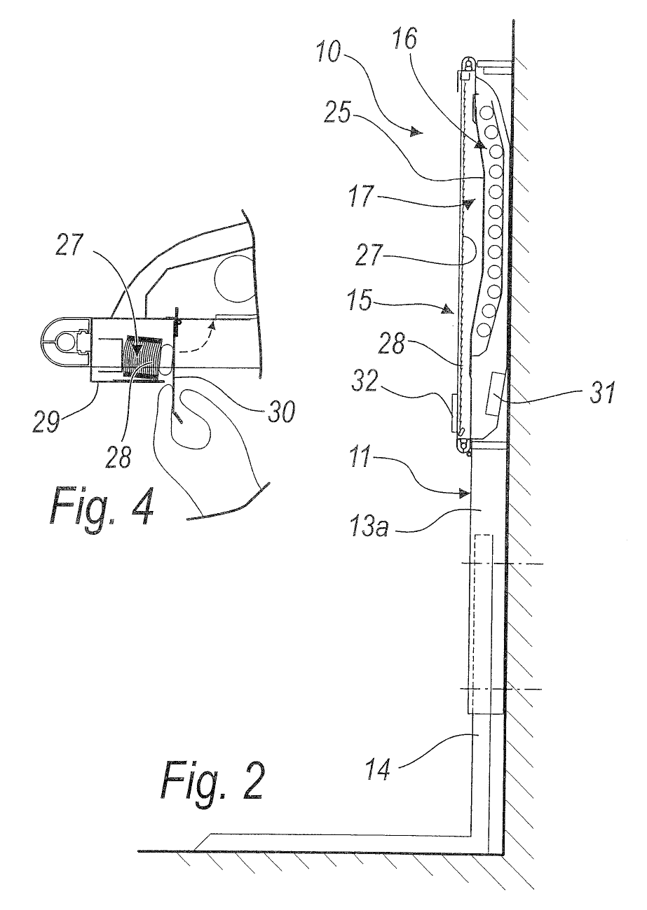

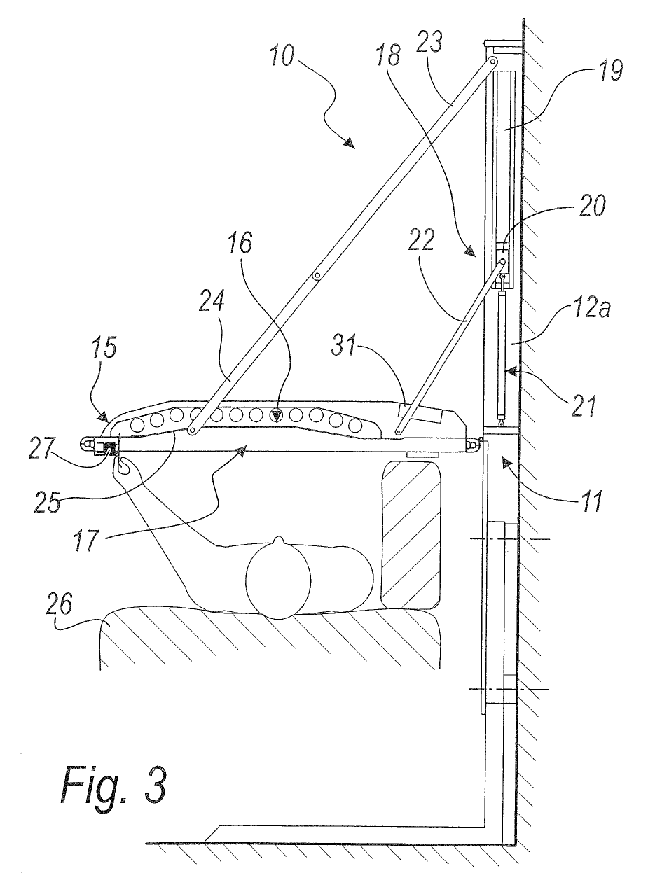

### WAYS OF CARRYING OUT THE INVENTION

With reference to the figures, a body tanning device according to the invention is generally designated by the reference numeral 10.

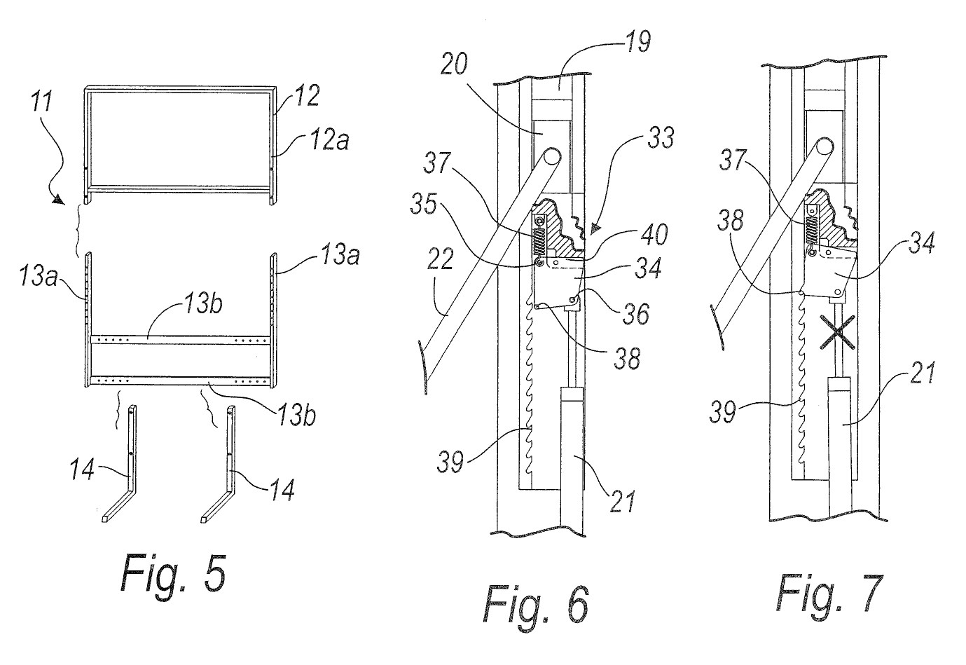

The device 10 comprises a chassis 11 for support on the floor and/or on a wall, which in this embodiment is constituted by a perimetric element 12 which is fixed to two uprights 13a connected by two cross-members 13b, as shown in FIG. 5.

Vertical portions 12a of the perimetric element 12 are tubular and allow the insertion of the uprights 13a.

In this embodiment, floor contact feet 14, each constituted by an L-shaped body, are fixed to the cross-members 13b.

Means for varying the height and width (not designated by reference numerals in the figures) are associated with the chassis 11 in order to achieve correct positioning of the device once installed.

In practice, these means for varying the height and width are constituted by removable connection elements (such as for example bolts), not shown in the Figures, which pass through corresponding holes formed in the tubular vertical portions 12a of the perimetric element 12 and through corresponding holes of a first series of adjustment openings provided along the uprights 13a; such means for varying height and width are also constituted by other removable connection elements, which pass through corresponding holes of a second series of adjustment openings provided along the cross-members 13b and two holes formed in the feet 14.

It is therefore possible to adjust the height of the perimetric element 12 with respect to the uprights 13a and the distance of the feet 14 on the cross-members 13b.

In this embodiment, the chassis 11 is also fixed to the wall, in order to increase the stability of said device (in other embodiments, the chassis can be fixed conveniently only to the wall, without supports on the floor).

To do this, there are means for fixing on the wall (not shown in the Figures), which are constituted for example by expansion elements which are inserted through the uprights 13a or the cross-members 13b and grip within the wall.

A frame 15 for supporting a source of tanning rays 16 is pivoted horizontally to the chassis 11.

In this embodiment, the tanning ray source 16 is constituted for example by a plurality of low-pressure fluorescent lamps.

In this embodiment, the frame 15 is substantially a flat box which has a large opening 17 for the exit of the tanning rays.

In other embodiments, the frame 15 can be for example merely a perimetric element for lateral containment.

As mentioned, the frame 15 is pivoted about a horizontal axis to the chassis 11, in order to assume at least two stable positions: a first active position is substantially horizontal and a second inactive position is substantially vertical and adjacent to the wall.

In order to move the frame 15 from this first stable position to the second stable position and vice versa, there are means 18 for moving and supporting the frame 15.

The movement and support means 18 for the frame 15 comprise, substantially for each side of the frame 15, two vertical guides 19, each arranged on a respective vertical portion 12a of the perimetric element 12.

A corresponding carriage 20 which can slide vertically is arranged in each vertical guide 19.

A corresponding elastic element 21 is rigidly coupled to the carriage 20 and contrasts the advancement of the carriage 20 in a downward direction.

In this embodiment, the elastic element 21 is constituted by a gas-assisted strut, which is arranged vertically and below the carriage 20.

A rod 22 is articulated at the end to the carriage 20 and in turn is articulated to the frame 15.

The movement and support means 18 further comprise two additional rods 23, which are mutually articulated at one end and are articulated, at the opposite ends, respectively to the chassis 11, in a point located above the guide 19 and the frame 15, in a forward position with respect to the articulation point of the rod 22 (a position which is further spaced from the pivoting axis with respect to the articulation point of the rod 22).

In practice, the two additional rods 23 are rods which determine the rotation stroke limit for the frame 15.

A variation (not shown in the figures) of the elastic element constituted by the gas-assisted strut is constituted for example by a helical spring which is arranged above the carriage.

As regards the tanning ray source, it has been mentioned that it is constituted by a plurality of low-pressure fluorescent lamps.

In this embodiment, the low-pressure lamps are integrated in a tanning canopy panel 25 of a substantially known type, which is recessed within the frame 15.

Advantageously, the distance of the panel 25 from the axis about which the frame is pivoted to the chassis is at least 20 centimeters.

In this manner, the device 10, when arranged horizontally, has the tanning ray source 16 sufficiently spaced from the wall so that it is not hindered by the part of the piece of furniture which can be arranged below said device, such as for example the back of a couch 26 or the headboard of a bed.

Advantageously, the device comprises, in association with the frame, a retractable cover 27, of the substantially fold-away type, for the tanning ray source 16.

The retractable cover 27 is designed to close, in a substantially reversible manner, the large opening 17 formed on the frame 15 to allow the exit of the tanning rays.

The retractability indeed consists in the possibility to retract the blind from a visible configuration to a configuration which is substantially scarcely visible or not visible at all.

The retractable cover 27 is constituted by a fold-up blind, preferably such as a Venetian blind 28.

In particular, in this case, the upper supporting points of the Venetian blind 28 are arranged inside the upper edge 29 of the metal plate of the frame 15 and therefore are hidden from view; advantageously, the blind 28, when it is fully retracted and compacted, if the frame 15 is arranged horizontally, is arranged so as to rest on the upper edge 29.

The entire Venetian blind 28 is arranged within the edge of the opening 17 formed by the frame 15.

Conveniently, in order to prevent the user from touching the Venetian blind 28 and ruining it during the movement of the frame 15, a longitudinal grip flap 30 is pivoted proximate to the upper edge 29, in an inner position thereof.

The longitudinal grip flap 30 is arranged within the chassis 11 and can be gripped, turning it outward, when the blind 28 is fully folded upward.

Variations of the retractable cover 27 (not shown in the Figures) can be for example a collapsible blind of the concertina type or for example a roll-up blind.

In the latter case, the roll-up blind can be constituted by a roller blind or by a shutter blind.

The retractable cover 27 has, when it is lowered so as to cover the tanning ray source, a series of decorative patterns in order to make the object pleasant to view.

Conveniently, a lighting lamp 31 is provided and recessed within the frame 15.

Moreover, spotlights 32 for reading are present and are fixed outside the frame 15.

The tanning device 10 further comprises safety means 33 for preventing the fall of the frame 15.

The safety means 33 for preventing the fall of the frame 15 comprise, for each carriage 20, a braking body 34, which in practice is a centrally-pivoted lever which is pivoted to the carriage 20 in a point which is substantially aligned with the vertical central axis of the carriage.

The braking body 34 is further articulated in two points thereof (which in this embodiment are not aligned with the point of articulation to the carriage 20), respectively an upper point 35 and a lower point 36, to the lower end of a spring 37, which is fixed at the opposite end to the carriage 20, and to the upper end of the stem of the gas-assisted strut 21.

On the braking body 34, in a position which lies above the lower point 36 of articulation of the gas-assisted strut 21, there is a portion 40, which abuts against the carriage 20 during the normal movement of the carriage 20.

An engagement element 38 extends from the braking body 34, in a position which is substantially opposite with respect to the portion 40 relative to the line connecting the upper and lower points 35 and 36, and is adapted to mate, when the frame 15 is in an uncontrolled fall, with respective locking abutments 39 formed along the guide 19.

In practice, during normal operation, the gas-assisted strut 21 pushes against the braking body 34, overcoming the force of the spring 37 and keeping the engagement element 38 outside the locking abutments 39; in particular, the braking body 34 pushes with the portion 40 against the carriage 20.

When, for some reason, the force of the gas-assisted strut 21 is lower than the value of the spring 37, for example due to the breakage of the stem of the gas-assisted strut 21, as shown in FIG. 6, the spring 37 turns the braking body 34, moving the engagement element 38 toward the locking abutments 39.

In practice it has been found that the invention thus described solves the problems described in known types of body tanning device; in particular, the present invention provides a body tanning device which can be accommodated conveniently in a domestic environment without interfering particularly with its aesthetics.

The device can in fact be arranged conveniently adjacent to a wall and above a piece of furniture, which can then be used as a lying surface for the user during tanning.

This is allowed by the fact that the height and width of the supporting chassis of the frame which supports the tanning source is adjustable both in height and width, so as not to interfere with the piece of furniture which will be adjacent to said chassis and will be arranged below the tanning panel once installed.

Moreover, this insertion in the domestic environment is allowed by the retractable covering which conceals the technological and functional particularities thereof.

The installation plan of the device entails positioning it rigidly, in association with the furniture that exists in the domestic environment, and strictly adjacent in a fixed manner to the wall of the room, substantially in one of these three typical situations: behind the headboard of a double bed, behind the back of a couch, and along the side of any bed.

As mentioned, the tanning technology is of the type with a low-pressure fluorescent lamp and the tanning screen is therefore large and very heavy.

Despite the considerable dimensions, conversion of the device is performed with a small number of simple operations, and such device, once closed, integrates harmoniously in the surrounding domestic environment.

In practice, the conversion consists in lifting the protruding side of the frame by turning the structure through approximately 90°; once rotation has occurred, said frame, with the tanning source inside, is arranged parallel to the wall, being flush-mounted and remaining in practice in its receptacle, retained in position for example by a magnet.

At this point, in order to complete the operation for concealing the tanning source it is sufficient to lower the retractable cover.

In practice, this conversion reduces significantly the space occupation of the device and changes its appearance into the appearance of a substantially ornamental object.

This type of structure is such that the elements that compose the tanning device are substantially concealed, making said device assume the appearance of a large furniture complement mounted on a support, thus avoiding the aesthetic impact that a tanning device of such size would have on a domestic environment, such as the living room or a bedroom, and thus allowing to install a tanning device in a permanent manner.

In this way, the user is in practice spared the inconvenience and risks of injury related to the need to move the tanning device at the end of each use.

Advantageously, the structure of the device entails that the radiating surface is correctly superimposed on the actual space where the user lies, since the presence of the headboard of the bed or of the back of the couch is taken into account; the starting point of the tanning surface is in fact at least 20 cm from the point where said frame is pivoted to the chassis.

The use of a lamp for lighting which is recessed within the frame allows to use the device, when not employed as a tanning device, as a lighting element which can produce a particular atmosphere.

In association with the use of a Venetian blind, by acting on the inclination of the slats of said blind, it is possible to adjust the luminous intensity emitted by said lamp, which protrudes from the frame.

Such lamp can be, for example, a colored fluorescent lamp.

The presence of spotlights outside the frame allows to use the device also as a lighting device for reading.

Another particularity of the invention is linked to the particular structure of the means for supporting and moving the frame.

In such means, the supports of said frame in fact lie substantially above the axis about which said frame is pivoted to the chassis.

In this manner, said movement means do not interfere with the presence of the headboard of the bed or of the back of the couch, thus allowing to arrange them directly adjacent to the frame, therefore compacting the device against the wall.

Advantageously, the particular mechanism studied to move and counterweight substantially the moving mass of the device is particularly effective, even if it is conceived in terms of traditional systems composed only of pneumatic springs associated with tanning canopies.

The system used to perform the manual movement of the tanning panel in a bed in a tanning canopy is in fact constituted by two pneumatic springs, which are applied to the ends of the long side of the canopy.

Said springs connect the canopy directly to the chassis, onto which they discharge their weight force, and are mounted in an inclined and lower position with respect to the weight that they must balance.

This configuration does not usually apply optimum balancing, since it is unable to follow the variations of the weight force due to the variation in the inclination of the rotating mass and the consequent displacement of the moving weights.

Other disadvantages in the use of pneumatic gas-assisted struts are linked to the thermal variations of the gas and to their loss of effectiveness due to wear.

The mechanism studied in the present invention is capable of applying a variable counterweight force, in practice a force which can balance the weight force of the frame in its movement along the entire extent of the rotation through 90° of said frame.

The invention thus conceived is susceptible of numerous modifications and variations, all of which are within the scope of the appended claims; all the details may further be replaced with other technically equivalent elements.

In practice, the materials employed, so long as they are compatible with the specific use, as well as the dimensions, may be any according to requirements and to the state of the art.

The disclosures in Italian Utility Model Application No. PD2005U000020 from which this application claims priority are incorporated herein by reference.

What is claimed is:

1-23. (canceled)

24. A body tanning device, comprising:
a chassis for support on the floor and/or on a wall,
a supporting frame for a tanning ray source, which has a large opening for the exit of said tanning rays, said frame being pivoted to said chassis in order to assume at least two stable positions: a first position for use, which is substantially horizontal, and a second substantially vertical position for nonuse,
means for moving and supporting said frame from said first position to said second position and vice versa,
further comprising a retractable covering, substantially of the fold-away type, for said tanning ray source, said covering being associated with said frame.

25. The device of claim 24, wherein said retractable covering is constituted by a collapsible blind.

26. The device of claim 25, wherein said collapsible blind is of the Venetian-blind type.

27. The device of claim 25, wherein said collapsible blind is of the concertina type.

28. The device of claim 24, wherein said retractable covering is constituted by a blind of the roll-up type.

29. The device of claim 28, wherein said retractable covering is constituted by a roller blind or by a shutter blind.

30. The device of claim 24, wherein said covering has decorative patterns exposed to view.

31. The device of claim 24, wherein said means for moving and supporting said frame are arranged substantially above the axis about which said frame is pivoted to said chassis.

32. The device of claim 31, wherein said movement and support means comprise:
at least one vertical guide, which is associated with said chassis,
a carriage, which is arranged so that it can slide within said at least one vertical guide,
an elastic element, which contrasts the advancement of said carriage in a downward direction,
a rod, which is articulated at its ends respectively to said carriage and to said frame.

33. The device of claim 32, wherein it comprises predominantly axially elongated means for supporting said frame, which are fixed at a first end to said frame in a position which is further spaced from the pivoting axis with respect to said rod and are rigidly coupled, at the opposite end, to said chassis in a position which lies above said guide.

34. The device of claim 33, wherein said predominantly axially elongated means for supporting said frame comprise two additional rods, which are mutually articulated at corresponding ends and are articulated, at the opposite ends, to said frame and to said chassis.

35. The device of claim 32, wherein said elastic element comprises a gas-assisted strut, which is arranged vertically and below said carriage.

36. The device of claim 32, wherein said elastic element is constituted by a metallic helical spring, which is arranged above said carriage.

37. The device of claim 24, wherein means for varying the height and width are associated with said chassis, in order to achieve correct positioning of the device once installed.

38. The device of claim 37, wherein said chassis comprises a perimetric element, which is fixed to at least two uprights, which are connected by at least one cross-member, the vertical portions of said perimetric element being tubular, thus allowing the insertion of said uprights.

39. The device of claim 38, wherein feet for resting on the ground are associated with said at least one cross-member.

40. The device of claim 39, wherein said means for varying the height and width of said chassis are constituted by removable connecting elements, which pass through corresponding holes formed in the tubular vertical portions of the perimetric element and in corresponding holes of a first series of adjustment openings provided along the uprights, said height and width variation means being also constituted by other removable connecting elements, which pass through corresponding holes of a second series of adjustment openings provided along the cross-members and a pair of holes formed in the feet.

41. The device of claim 24, wherein means for fixing to a wall are associated to said chassis.

42. The device of claim 24, wherein said tanning ray source is constituted by a tanning canopy panel, which is recessed within said frame, the distance of said panel from the axis for pivoting to said chassis being at least 20 cm.

43. The device of claim 24, further comprising a lighting lamp, which is recessed within said frame.

44. The device of claim 24, further comprising at least one spotlight, which is associated outside said frame.

45. The device of claim 24, wherein it comprises safety means for preventing the fall of said frame.

46. The device of claim 45, wherein said safety means for preventing the fall of said frame comprise, for at least one said carriage, a braking body, which is a centrally-pivoted lever which is pivoted to said carriage in a point which is substantially aligned with the vertical central axis of the carriage, said braking body being further articulated in two of its points, respectively an upper point and a lower point, to the lower end of a spring, which is fixed at the opposite end to said carriage, and with the upper end of said elastic element, on said braking body, in a position which lies above said lower point, a portion for abutment against said carriage during the normal movement of said carriage being provided, an engagement element protruding from said braking body, in a position which is substantially opposite said portion with respect to the connecting line of said upper and lower points, and being adapted to mate, when said frame is in an uncontrolled fall, with respective locking abutments formed along said guide.
